(19) United States
(12) Patent Application Publication
(10) Pub. No.: US 2016/0256509 A1
(43) Pub. Date: Sep. 8, 2016

(54) Vasodilator Formulation and Method of Use

(71) Applicant: ATP INSTITUTE PTY LTD, Loganholme, Queensland (AU)

(72) Inventor: Matthew Legge, Loganholme, Queensland (AU)

(73) Assignee: ATP Institute Pty. Ltd., Loganholme, Queensland (AU)

(21) Appl. No.: 15/032,370

(22) PCT Filed: Nov. 4, 2014
(86) PCT No.: PCT/AU14/50333

§ 371 Date: Apr. 27, 2016

(30) Foreign Application Priority Data
Nov. 4, 2013 (AU) 2013904256

(51) Int. Cl. A61K 36/67; A61K 36/534; A61K 36/53; A61K 8/06; A61K 9/00; A61K 47/46; A61K 47/12; A61K 8/97; A61K 8/67; A61K 9/107; A61K 36/185; A61K 31/355; A61K 31/198; A61K 45/06; A61Q 7/00; A61Q 19/08; A61K 8/44

(52) U.S. Cl. CPC A61K 36/67; A61K 8/44; A61K 36/534; A61K 36/53; A61K 8/062; A61K 9/0014; A61K 47/46; A61K 47/12; A61K 8/97; A61K 8/678; A61K 9/107; A61K 36/185; A61K 31/355; A61K 31/198; A61K 45/06; A61Q 7/00; A61Q 19/08; A61K 2800/592

(57) ABSTRACT

A topically admimstrable vasodilator formulation comprises arginine and/or one or more derivatives thereof, black pepper extract and/or one or more components or derivatives thereof; and peppermint extract and/or one or more components or derivatives thereof. The formulation may further comprise rosemary oil, a penetration enhancers, an ecdysteroid and/or one or more other agents that enhance the vasodilator activity of the formulation. The valodilator formulation may be suitable for treating or preventing a disease, disorder or condition associated with a decrease and/or impairment of blood flow in a subject by topically administration to the subject.

### TECHNICAL FIELD

THIS INVENTION relates to topical vasodilator formulations and methods of treatment and/or use. The formulations are useful for a variety of cosmetic and/or therapeutic applications such as for example enhancing muscular definition and to assist in the therapeutic treatment of diabetes.

### BACKGROUND

Approaches to improving blood flow to the skin have been many and consist of both systemic and topical approaches. Many beneficial effects can be experienced by a subject through an improvement in local blood flow, since impairment of local blood flow causes a variety of negative consequences. Among these are cold hands and feet, baldness, leg ulcers, certain forms of impotence, as well as a variety of other ailments.

Arginine is a naturally occurring amino acid, which participates in many important biochemical reactions essential to the normal physiology of a subject. This amino acid is found in most proteins consumed in a human's daily diet and can be metabolized to support glucose synthesis or catabolised to produce energy.

While many of the currently available topical vasodilator formulations increase or enhance blood flow to some degree, there is nonetheless, a continued interest in identifying new formulations, which provide longer lasting vasodilator effects without undesirable side effects.

Accordingly, there is continued interest in the development of new topical vasodilator formulations.

### SUMMARY

The present invention is directed to formulations and methods of increasing or enhancing blood flow in a subject.

In a broad form, the invention relates to vasodilator formulations, for increasing, enhancing and/or stimulating blood flow to assist in the therapeutic treatment of diseases and/or conditions such as for example diabetes and in cosmetic applications such as for example the enhancement of muscle and vein definition in body builders, physique models, models, and/or aesthetically conscious individuals.

In a first aspect, the invention provides a topically administrable vasodilator formulation comprising
(i) arginine and/or one or more derivatives thereof;
(ii) black pepper extract and/or one or more components or derivatives thereof; and
(iii) peppermint extract and/or one or more components or derivatives thereof.

In one embodiment, arginine and/or one or more derivatives thereof is selected from the group consisting of: 2-Amino-5-guanidinopentanoic acid, agmatine, arginine hydrochloride, Ark 1, decarboxylated arginine, dipeptide arginyl aspartate, D-arginine, L-arg, L-arginine, L-arginine aspartate, NG-monomethyl-L-arginine, arginine alpha ketogluterate, arginine-ethyl esther, norvaline, arginine salt, arginine ester, Argivene, Detoxargin, Levargin, Minophagen Argamine, Polyarginine, Arginina, (S)-2-Amino-5-guanidinopentanoic acid, 2-amino-5-guanidinovaleric acid, Argininum [INN-Latin], Arginina [INN-Spanish], L-alpha-Amino-delta-guanidinovaleric acid, L-Arginin, Poly(L-arginine), L-Ornithine, N5-(aminoiminomethyl)-, (L+)-Arginine, 1-Amino-4-guanidovaleric acid, H-Arg-OH, L-a-Amino-d-guanidinovaleric acid, L-Arginine, homopolymer, (S)-2-Amino-5-[(aminoiminomethyl)amino]pentanoic acid, L-Arginine hydrochloride, L-Norvaline, 5-((aminoiminomethyl)amino)-, (S)-2-Amino-5-guanidinovaleric acid, (2S)-2-amino-5-(diaminomethylideneamino)pentanoic acid, (S)-2-Amino-5-((aminoiminomethyl)amino)pentanoic acid, Pentanoic acid, 2-amino-5-((aminoiminomethyl)amino, L-Norvaline, 5-[(aminoiminomethyl)amino]-2-Amino-5-Guanidnovaleric Acid, Argininum Pentanoic acid, 2-amino-5-[(aminoiminomethyl)amino]-, Tocris-0663 L-Arginine (JP16), Lopac-A-5006, Arginine, 2-amino-5-guanidinovalerate, Arginine hydrochloride (USAN), L-a-Amino-d-guanidinovalerate, N5-(aminoiminomethyl)-L-Ornithine, L-alpha-Amino-delta-guanidinovalerate, (2S)-2-amino-5-guanidinopentanoic acid, 5-[(aminoiminomethyl)amino]-L-Norvaline, (2S)-2-amino-5-(carbamimidamido)pentanoic acid, L-Arginine-L-Glutamate 2-Amino-Pentanedoic Acid, (S)-2-amino-5-[(aminoiminomethyl)amino]-Pentanoate, (S)-2-amino-5-[(aminoiminomethyl)amino]-Pentanoic acid, (2S)-2-azanyl -5-[bis(azanyl)methylideneamino]pentanoic acid, alpha-keto-gamma-guanidovaleric acid, ornithine, citrulline, guanidoacetic acid and ornithine.

Preferably, the vasodilator formulation comprises arginine hydrochloride.

In one embodiment the vasodilator formulation comprises from about 0.1% to 75% arginine and/or one or more derivatives thereof. Preferably, the vasodilator formulation comprises about 2.5% arginine and/or one or more derivatives thereof. More preferably, the vasodilator formulation comprises about 2.5% arginine hydrochloride.

Suitably, the formulation comprises black pepper extract and/or one or more components or derivatives thereof at a concentration of about 0.01 to 25%. Preferably, the formulation comprises black pepper extract and/or one or more components or derivatives thereof at a concentration of about 0.25%.

In one embodiment, black pepper extract and/or one or more components or derivatives thereof is black pepper oil.

Suitably, the formulation comprises peppermint extract and/or one or more components or derivatives thereof at a concentration of about 0.01 to 25%. Preferably, the formulation comprises peppermint extract and/or one or more components or derivatives thereof at a concentration of about 0.25%.

In one embodiment, peppermint extract and/or one or more components or derivatives thereof is peppermint oil.

In one embodiment. the topical formulation further comprises rosemary oil and/or one or more components or derivatives thereof. Preferably, the formulation comprises rosemary oil and/or one or more components or derivatives thereof at a concentration of about 0.01 to 50% or preferably at a concentration of about 5-10%.

In one embodiment, the vasodilator formulation further comprises one or more penetration enhancers.

In one embodiment, the penetration enhancer is aloe vera extract or one or more components or derivatives thereof and/or a fatty acid penetration enhancer. Preferably, the fatty acid penetration enhancer is stearic acid.

In one embodiment, the formulation comprises stearic acid at a concentration from about 1 to 75%. Preferably, the formulation comprises stearic acid at a concentration of about 5%.

Suitably, the formulation comprises aloe vera extract or one or more components or derivatives thereof at a concentration of about 0.01 to 20%. Preferably, the formulation comprises Aloe Barbadensis Leaf Juice or one or more components or derivatives thereof. Suitably, the Aloe Barbadensis Leaf Juice is present in the formulation at a concentration of about 2%.

In one embodiment, the Aloe Barbadensis Leaf Juice or one or more components or derivatives thereof is peppermint oil.

In one embodiment, the formulation may further comprise at least one terpene. Preferably, the terpene is from black pepper (Piper nigrum) extract or one or more components or derivatives thereof and/or peppermint extract or one or more components or derivatives thereof. More preferably, the terpene is from black pepper and/or peppermint oil.

In one embodiment, the topical formulation further comprises one or more ecdysteroids.

In one embodiment, the topical formulation further comprises one or more antimicrobials.

The topical vasodilator formulation may further comprise one or more alpha-2 antagonists.

In one embodiment, the topical formulation further comprises one or more phosphodiesterase-5a inhibitors.

In one embodiment, the topical formulation further comprises lysine.

The topical vasodilator formulation may further comprise one or more alpha reductase inhibitors.

In one embodiment, the topical formulation further comprises one or more hair growth promoters.

In one embodiment, the topical formulation further comprises one or more immune and/or cytokine modulators. The topical vasodilator formulation may further comprise one or more additional vasodilators.

In one embodiment the additional vasodilators comprise nitric oxide based vasodilators. Suitably, the nitric oxide based vasodilators include for example Nitroglycerin, amyl nitrite, isobutyl nitrite, sodium nitroprusside, S-nitroso-N-acetylpenicillamine, isosorbide dinitrate, sildenafil, tetrahydrobiopterin, spironolactone, nitrites and nitrates

In one embodiment, the topical formulation further comprises Hippophae rhamnoides extract or one or more components or derivatives thereof.

In one embodiment, the topical vasodilator formulation further comprises at least one pharmaceutically acceptable carrier, diluent and/or excipient. Preferably, at least one other pharmaceutically acceptable carrier, diluent and/or excipient may include one or more of a solubiliser, emollient, moisturiser, thickener, skin conditioner, preservative and/or stabiliser as would be understood by a person of skill in the art.

In one embodiment, the pharmaceutically acceptable carrier is an oil-in-water emulsion. Suitably, the oil-in-water emulsion is a low oil, high water emulsion.

In one embodiment, the pharmaceutically acceptable carrier, diluent and/or excipient further comprises an emulsifying wax. Preferably, the emulsifying wax is Cetearyl alcohol and/or Ceteareth-20.

In one embodiment, the topical formulation is provided as an oil, mousse, gel, cream, lotion, foam, balm, ointment, liniment, liquid, aerosol, self-tanning lotion, oil, spray or paint and the like.

Preferably, the topical formulation is a cream or lotion.

In one embodiment, the present invention provides a formulation in the form of a vasodilator enhancer cream for topical application. The formulation comprises arginine, black pepper extract, peppermint extract, a skin penetration enhancer, aqua, natural emulsifying wax, ethyl-alcohol, ecdysterone, sorbitol, aloe barbadensis leaf juice, stearic acid, cyclopentasiloxane, tocopheryl, phenoxyethanol and caprylyl Glycol.

In a second aspect, the invention provides a method of producing the topically administrable vasodilator formulation of the first aspect, including the step of combining arginine and/or one or more derivatives thereof; black pepper extract and/or one or more components or derivatives thereof; and peppermint extract and/or one or more components or derivatives thereof to thereby produce the topically administrable vasodilator formulation.

The method of this aspect may further include combining one or more other ingredients or components of the formulation set forth in the first aspect.

In one embodiment, one or more of the components or ingredients of the formulation are micronized.

In one embodiment, the dry ingredient is at least one arginine or derivatives thereof. In one embodiment the dry ingredient is one or more ecdysteroids.

In a third aspect, the invention provides a method of treating or preventing a disease, disorder or condition associated with a decrease and/or impairment of blood flow in a subject, said method including the step of topically administering to said subject an effective amount of a topical vasodilator formulation according to the first aspect or produced according to the method of the second aspect, to increase, enhance and/or stimulate blood flow and/or vasodilation in the subject.

In a fourth aspect, the invention provides a topically administrable vasodilator formulation according to the first aspect or produced according to the method of the second aspect for use in the therapeutic and/or prophylactic treatment of a disease, disorder or condition associated with a decrease and/or impairment of blood flow and/or vasoconstriction in a subject.

In one embodiment, the topical vasodilator formulation is topically administered to a subject at a location proximal to said decrease and/or impairment of blood flow.

In one embodiment, the disease, disorder or condition associated with a decreased or impaired blood flow and/or vasoconstriction is selected from the following non-limiting examples, including erectile dysfunction, aging, baldness, peripheral neuropathy, microangiopathy, female sexual dysfunction, male sexual dysfunction, diabetes, aging, restless leg syndrome, raynaud's phenomenon, Buerger's Disease, chilblains, numbness and tingling of extremities, varicose veins, haemorrhoids, hypothyroidism, immobility, cellulite, accumulation of subcutaneous adipose tissue, cosmetic applications such as poor quality hair, nail and skin, lymphedema, swelling of the hands and feet oedema, deep vein thrombosis, ischemia, chronic venous insufficiency, gangrene, vasoconstriction, thrombosis, embolism, paraesthesia, poikilothermia, cellulitis, tissue necrosis, ischaemic neuropathy, leg cramps either idiopathic, or related to either pregnancy, renal dialysis, or peripheral vascular disease (both venous and arterial), or to revitalize muscle, or to improve muscle strength and recovery after vigorous exercise and intense sport, or to improve muscle strength and performance during vigorous exercise and intense sport.

In one embodiment, the subject is a mammal Preferably the subject is a human.

Alternatively, the subject is a non-human mammal, non-limiting examples of which include a. horse, dog, cat, rabbit and the like.

In a fifth aspect, the invention provides a method of enhancing muscular and/or vascular definition in a subject, the method including the step of topically administering to said subject an effective amount of a topical vasodilator formulation according to the first aspect or produced according to the method of the second aspect, to enhance muscular and/or vascular definition in the subject.

In one embodiment the subject is a body builder, physique model, model and/or an aesthetically conscious individual.

In a sixth aspect, the invention provides a kit comprising the topically administrable vasodilator formulation according to the first aspect or produced according to the method of the second aspect, an applicator device and instructions for using said formulation to increase, enhance and/or stimulate blood flow and/or vasodilation in a subject.

In one embodiment, the applicator device is pump device.

Throughout this specification, unless otherwise indicated, “comprise”, “comprises” and “comprising” are used inclusively rather than exclusively, so that a stated integer or group of integers may include one or more other non-stated integers or groups of integers.

As used in this specification the indefinite articles “a” and “an” may refer to one entity or a plurality of entities and are not to be read or understood as being limited to a single entity.

### DETAILED DESCRIPTION

The present inventors have created an improved formulation which, when applied to the skin can effectively enhance blood flow and/or vasodilation when delivered to a target skin site.

Effective concentrations of the amino acid arginine and/or derivatives thereof, black pepper extract or one or more components or derivatives thereof, and peppermint extract or one or more components or derivatives thereof can be formulated to be topically applied.

The present invention provides a formulation and method for prophylactically or therapeutically treating a disease, disorder or condition associated with impaired blood flow and/or vasoconstriction or enhancing muscular and/or vascular definition in a subject,

### Vasodilator Topical Formulations

In a first aspect, the invention provides a topically administrable vasodilator formulation comprising:
(i) arginine and/or one or more derivatives thereof;
(ii) black pepper extract and/or one or more components or derivatives thereof and
(iii) peppermint extract and/or one or more components or derivatives thereof.

As used herein, the term “extract” refers to a composition or preparation comprising one or more active components, compounds or substances obtained, isolated or extracted from a particular source. The active components, compounds or substances in the extract may be in a more concentrated or enriched form compared to the source. In particular, the extract may be obtainable from a plant or any portion thereof, including for example peppermint.

The term “derivative” refers to a modified form of a particular compound or substance. The derivative may be a modified form of a compound or substance that is a component of the amino acid arginine for example. Typically, the derivative is a chemically modified or related form of the particular compound or substance,

As used herein, “% concentration” may refer to percent weight/volume (w/v), percent weight/weight (w/w) or percent volume/volume (v/v) of a particular ingredient within the formulation as applicable.

In one embodiment, the topical vasodilator formulation comprises from about 0.1% to 75% arginine and/or one or more derivatives thereof.

Suitably, the formulation comprises at least one arginine or one or more derivatives thereof at a concentration from about 0.1, 0.2. 0,3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5 to 75%.

Preferably, the vasodilator formulation comprises about 2.5% arginine and/or one or more derivatives thereof.

In one embodiment, arginine is selected from the group consisting of: 2-Amino-5-guanidinopentanoic acid, agmatine, arginine hydrochloride, Ark 1, decarboxylated arginine, dipeptide arginyl aspartate, D-arginine, L-arg, L-arginine, L-arginine aspartate, NG-monomethyl-L-arginine, arginine alpha ketogluterate, arginine-ethyl esther, norvaline, arginine salt, arginine ester, Argivene, Detoxargin, Levargin, Minophagen Argamine, Polyarginine, Arginina, (S)-2-Amino-5-guanidinopentanoic acid, 2-amino-5-guanidinovaleric acid, Argininum [INN-Latin], Arginina [INN-Spanish], L-alpha-Amino-delta-guanidinovaleric acid, L-Arginin, Poly(L-arginine), L-Ornithine, N5-(aminoiminomethyl)-, L(+)-Arginine, 1-Amino-4-guanidovaleric acid, H-Arg-OH, L-a-Amino-d-guanidinovaleric acid, L-Arginine, homopolymer, (S)-2-Amino-5-[(aminoiminomethyl)amino]pentanoic acid, L-Arginine hydrochloride, L-Norvaline, 5-((aminoiminomethyl)amino)-, (S)-2-Amino-5-guanidinovaleric acid, (2S)-2-amino-5-(diaminomethylideneamino)pentanoic acid, (S)-2-Amino-5-((aminoiminomethyl)amino)pentanoic acid, Pentanoic acid, 2-amino-5-((aminoiminomethyl)amino, L-Norvaline, 5-[(aminoiminomethyl)amino]-2-Amino-5-Guanidnovaleric Acid, Argininum Pentanoic acid, 2-amino-5-[(aminoiminomethy)amino]-, Tocris-0663, L-Arginine (JP16), Lopac-A-5006, Arginine, 2-amino-5-guanidinovalerate, Arginine hydrochloride (USAN), L-a-Amino-d-guanidinovalerate, N5-(aminoiminomethyl)-L-Ornithine, L-alpha-Amino-delta-guanidinovalerate, (2S)-2-amino-5-guanidinopentanoic acid, 5-[(aminoiminomethyl)amino]-L-Norvaline, (2S)-2-amino-5-(carbamimidamido)pentanoic acid, L-Arginine-L-Glutamate 2-Amino-Pentanedoic Acid, (S)-2-amino-5-[(aminoiminomethyl)amino]-Pentanoate, (S)-2-amino-5-[(aminoiminomethyl)amino]-Pentanoic acid, (2S)-2-azanyl-5-[bis(azanyl)methylideneamino]pentanoic acid, alpha-keto-gamma-guanidovaleric acid, ornithine, citrulline, guanidoacetic acid and ornithine.

In one embodiment, the formulation comprises a combination of arginine and one or more derivatives thereof. Alternatively, the formulation comprises arginine or one or more derivatives thereof.

Preferably, the formulation comprises arginine hydrochloride.

Suitably, the formulation comprises black pepper extract and/or one or more components or derivatives thereof at a concentration of about 0.01 to 25%.

In one embodiment, the formulation comprises black pepper extract and/or one or more components or derivatives thereof at a concentration from about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5. 14, 14.5, 15, 15.5, 16, 16.5, 17, 17.5, 18, 18.5, 19, 19.5, 20, 20.5, 21, 21.5, 22, 22.5, 23, 23.5, 24. 24.5 to 25%.

Preferably, the formulation comprises black pepper extract and/or one or more components or derivatives thereof at a concentration of about 0.25%.

In one embodiment, the black pepper extract and/or one or more components or derivatives thereof is black pepper oil.

In one embodiment, the formulation comprises a combination of black pepper extract and one or more components or derivatives thereof. Alternatively, the formulation comprises black pepper extract or one or more components or derivatives thereof.

Suitably, the formulation comprises peppermint extract and/or one or more components or derivatives thereof at a concentration of about 0.01 to 25%.

In one embodiment, the formulation comprises peppermint extract and/or one or more components or derivatives thereof at a concentration from about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5 to 25%.

Preferably, the formulation comprises peppermint extract and/or one or more components or derivatives thereof at a concentration of about 0.25%.

In one embodiment, the peppermint extract and/or one or more components or derivatives thereof is peppermint oil.

In one embodiment, the formulation comprises a combination of peppermint extract and one or more components or derivatives thereof. Alternatively, the formulation comprises peppermint extract or one or more components or derivatives thereof.

In one embodiment, the topical formulation further comprises rosemary oil. As used herein “rosemary” may include species such as Rosmarinus officinalis and Rosmarinus coronarium, although without limitation thereto. The oil may be an essential oil an infused oil or any other lipid-containing extract, fraction or infusion that comprises one or more therapeutically effective elements of rosemary.

Suitably, the rosemary oil is at a concentration ranging from about 0.01% to 50%, preferably about 0.05% to 25%, more preferably about 1% to 20%, even more preferably about 2% to 15%, or advantageously about 5-10%, inclusive of 6%, 7%, 8% and 9%.

Rosemary oil may improve the circulatory stimulant activity of the formulation disclosed herein.

The vasodilator formulation may further comprise one or more penetration enhancers to aid penetration of the active ingredients.

Suitably, penetration enhancers include without limitation: Oleic acid, 2 N-nonyl-1,3-dioxolanes, N-acetyle prolinate esters (such as pentyl- and octyl-N-acetyle prolinate), alkyldiloxanes (e.g., 1-Alkyl-3-β-D glucopyranosyl-1,1,3,3-tetramethyl disiloxanes), transcarbam (such as 5-(dodecyloxycarbonyl)pentylammonium-5-(dodecyloxycarbonyl)pentylcarbamate), iminosulfurane (like N-hexyl, N-benzoyl-S,S-dimethylimino-sulfuranes), capsaicin derivatives (e.g., Nonivamide), cinnamene compounds (such as cinnamic acid, cinnamaldehyde etc), terpenes, fatty alcohol, pyrrolidone, sulfoxides, laurocapram, surface active agents, amides, amines, lecithin, polyols, quaternary ammonium compounds, silicones, alkanoates and cardamom seed.

In one embodiment, the penetration enhancer is aloe vera extract or one or more components or derivatives thereof and/or a fatty acid penetration enhancer. Preferably, the fatty acid penetration enhancer is stearic acid.

Suitably, the formulation comprises aloe vera extract or one or more components or derivatives thereof at a concentration of about 0.01 to 20%. Preferably, the formulation comprises Aloe Barbadensis Leaf Juice or one or more components or derivatives thereof. Suitably, the Aloe Barbadensis Leaf Juice is present in the formulation at a concentration of about 2%.

In one embodiment, the Aloe Barbadensis Leaf Juice or one or more components or derivatives thereof is peppermint oil.

In one embodiment, the formulation comprises stearic acid at a concentration from about 1 to 75%. Preferably, the formulation comprises stearic acid at a concentration of about 5%.

Alternate fatty acid penetration enhancers include for example: isostearic acid, lauric acid, myristic acid, capric acid, oleic, linoleic, linolenic acid, 1-carvyl esters, caproic acid/hexanoic acid, alkanoic acids, diacid, ethyloctadecanoic acid, lactic acid, linolaidic acid, neodecanoic acid, palmitic acid, pelargonic acid, propionic acid and vaccenic acid.

In one embodiment, the formulation may further comprise at least one terpene. Preferably, the terpene is from black pepper (Piper nigrum) extract or one or more components or derivatives thereof and/or peppermint extract or one or more components or derivatives thereof. More preferably, the terpene is from black pepper and/or peppermint essential oils.

In one embodiment, the topical vasodilator formulation further comprises an ingredient selected from the group consisting of: phytoecdysterones and ecdysones (e g., ecdysteroids), antimicrobials, alpha-2 antagonists, phosphodiesterase-5a inhibitors, lysine, 5-alpha reductase inhibitors, hair growth promoters, immune and cytokine modulators, additional vasodilators and Hippophae rhamnoides extract or one or more components or derivatives thereof.

Ecdysteroids for inclusion in the formulation may be selected and/or sourced from the following non-limiting examples: cyanatis vagas, Serratula, Silene species, Quinoa, chestnut and Leuzea, Suma extract, pfaffia extract, Brazilian ginseng extract, beta-ecdysterone, turkesterone, ecdysterone, Asparagus Filicinus, Spinacia oleracea, yams, white button Mushrooms, Ajuga Turkestanica, Rhaponticum carthamoides, Silene Praemixta, Vitex Scabra, as well as other Vitex species such as cymosa and canescens. Ecdysone, Ecdysterone, Beta-ecdysterone, 20-hydroxyecdysone, Turkesterone, Integristerone A, 24(28)-dehydramakisterone A, Viticosterone E, Sileneoside A and C, Ponasterone A, Cyasterone, 11-α-hydroxypoststerone, 9,11-Didehydropoststerone, Dacryhainansterone, 25-Hydroxydacryhainansterone, 14-Epi-20E, 24(28)-Dehydromakisterone A, 2-Deoxy-20E, 2-Deoxyecdysone, 2-Deoxyecdysone-22-acetate, Ajugasterone and Polypodine.

In one embodiment, the formulation comprises ecdysteroids at a concentration of about 5%. Suitably, the formulation may comprise the ecdysteroid 20-hydroxyecdysone at a concentration from about 1% to 50%. Preferably, the formulation may comprise 20-hydroxyecdysone at a concentration of about 5%.

Phytoecdysterones are plant based structural analogs of the insect moulting hormone ecdysone. Phytoecdysteroids and ecdysones are commonly referred to as ecdysteroids Ecdysteroids may serve as effective anabolic, hepatoprotective, immunoprotective, antioxidant and hypoglycemic agents. Ecdysteroids are general tonic and broad spectrum stimulants.

Antimicrobial ingredients for inclusion in the formulation may be selected from the following non-limiting examples: Kunzea ambigua extract or one or more components or derivatives thereof, eucalyptus extract or one or more components or derivatives thereof, Tea tree extract or one or more components or derivatives thereof, thyme extract or one or more components or derivatives thereof, Lavender extract or one or more components or derivatives thereof. Lemon extract or one or more components or derivatives thereof, Lemongrass extract or one or more components or derivatives thereof, Cinnamon extract or one or more components or derivatives thereof, Grapefruit extract or one or more components or derivatives thereof, Clove Bud extract or one or more components or derivatives thereof, Sandalwood extract or one or more components or derivatives thereof and Peppermint extract or one or more components or derivatives thereof.

In one embodiment, Lysine is added to the formulation. Preferably, the formulation further comprises Lysine at a concentration of about 0.5% to about 20%

Alpha-2 antagonists for inclusion in the formulation may be selected from the following non-limiting examples: Yohimbine/rauwaoliscine, Aspidosperma quebracho-blanco extract or one or more components or derivatives thereof, Yohimbine Hcl, Yohimbe and pharmaceuticals atipamezole, efaroxan and idazoxan.

Phosphodiesterase-5a inhibitors for inclusion in the formulation may be selected from the following non-limiting examples: quercetin or its analogues thereof, icariin contained in Epimedium grandiflorum, 4-Methylpiperazine and Pyrazolo Pyrimidin-7-1, components of the lichen Xanthoparmelia scabrosa, red onion peel extract or one or more components or derivatives thereof. Eurycomanone and other quassinoids (commonly found in Eurycoma longifolia), or pharmaceuticals, Sildenafil citrate, tadalafil and vardenafil,

5-alpha reductase inhibitors for inclusion in the formulation may be selected from the following non-limiting examples: Saw Palmetto (serenoa repens) extract or one or more components or derivative thereof, nettle (urtica dioica) root extract or one or more components or derivatives thereof containing 3,4-divanillyltetrahydrofuran, hippophae rhamnoides, epilobium, Finasteride, Dutasteride and Alfatradiol.

In one embodiment, the formulation comprises 5-alpha reductase inhibitors at a concentration from about 0.5% to 25%. Preferably, the formulation comprises 5-alpha reductase inhibitors at a concentration of about 2.5%.

Hair growth promoters for inclusion in the formulation may be selected from the following non-limiting examples: Eclipta alba, Crinum asiaticum and L-ascorbate-2-phosphate.

Additional vasodilators for inclusion in the formulation may be selected from the following non-limiting examples: vitamin B3 niacinamide, nicotinic acid, methyl nicotinate, Sea Buckthorn (Hippophae rhamnoides) extract or one or more components or derivatives thereof and nitric oxide based vasodilators.

Suitably, the nitric oxide based vasodilators include for example Nitroglycerin, amyl nitrite, isobutyl nitrite, sodium nitroprusside, S-nitroso-N-acetylpenicillamine, isosorbide dinitrate, sildenafil, tetrahydrobiopterin, spironolactone, nitrites and nitrates.

Immune and/or cytokine modulators for inclusion in the formulation may be selected from the following non-limiting examples: Vitamin D, polypodium leucotomas, tocopherols and omega 3, 6, 7 and/or 9 oils.

Hippophae rhamnoides for inclusion in the formulation may be selected from the following non-limiting examples: Sea Buckthorn (Hippophae rhamnoides) extract or one or more components or derivatives thereof, in particular, Sea Buckthorn oil.

The topical formulation of the invention may further comprise a pharmaceutically acceptable carrier, diluent or excipient. These include without limitation an effective amount of a moisturising, solubilising and/or substantive ingredient. A useful reference describing pharmaceutically acceptable carriers, diluents and excipients is Remington's Pharmaceutical Sciences (Mack Publishing Co NJ USA, 1991).

In one embodiment, the carrier is an oil-in-water emulsion. Suitably, the oil-in-water emulsion is a low oil, high water emulsion. More suitably, the oil:water ratio is from about 0.01 to 1 part oil to 1 part water. Preferably, the formulation is paraben free.

In addition to the above carrier, the formulation may also comprise Terpenes (Aqil, M et al., 2007, Drug Discovery Today, Vol 12, Issues 23-24, Pages 1061-1067; and Williams, A. C., and Barry, B. W., 1991, Pharmaceutical Research, vol. 8, Issue 1, Pages 17-24).

In one embodiment, terpenes include without limitation the following examples: Hemiterpenes, Monoterpenes, Sesquiterpenes, Diterpenes, Sesterterpenes, Triterpenes, Tetraterpenes, Polyterpenes. Alternatives may include Geraniol, Citronellol, Camphor, Pinenes (α and β)—Pine genera. Borneol, Rutaceae; Myrtaceae; Umbellifereae; Labiatae; Compositae; Pinaceae oils. Bergamot, Citronella, Laurel, Vetiver, Ginger, Sandalwood, Cinnamon, Nutmeg, cannabis sativa, Mentha×piperita, Mentha canadensis, Mentha spicata L. (M. viridis Linn.) and Mentha×cardiac, Mentha arvensis, oils are derived from Eucalyptus polybractea, Eucalyptus smithii, Eucalyptus australiana and Eucalyptus globulus.

Preferably, the terpenes in the formulation include black pepper extract or one or more components or derivatives thereof and menthol. More preferably, black pepper extract is black pepper oil. Suitably, black pepper extract is black pepper essential oil.

Suitably, black pepper essential oil comprises Limonene, Pinene, Myrcene, Phellandrene, Beta-caryophyllene, Beta-bisabolene, Sabinene, Pinocarveol, Alpha Terpineol, Camphene and Alpha Terpenene.

In one embodiment, the formulation comprises black pepper oil at a concentration from about 0.01 to about 25%. Preferably, the black pepper oil is at a concentration of about 0.25%.

Terpenes are included in the list of Generally Recognized As Safe (GRAS) substances and have low irritancy potential. Their mechanism of percutaneous permeation enhancement involves increasing the solubility of drugs in skin lipids, disruption of lipid/protein organization and/or extraction of skin micro constituents that are responsible for maintenance of barrier status.

Terpenes are compounds naturally found in many essential oils including those from black pepper. Terpenes can enhance the permeation of both hydrophilic and lipophilic drugs. Black pepper essential oil induces a warming sensation when applied to the skin due to local dilation of microcirculation to the skin, which is capable of enhancing percutaneous absorption of the active ingredients,

In some embodiments, the vasodilator formulation has a semi-solid consistency of a mousse, gel, cream, lotion, self-tanning lotion, oil, spray or paint for ease of storage and application.

The vasodilator formulation preferably has the consistency of a cream.

In one embodiment, an effective amount of a thickener may be incorporated within the formulation to obtain the desired viscosity and consistency of the product for example, as use as a cream, lotion or ointment.

Suitable thickening agents include acrylate polymers and co-polymers among the classes of polyacrylates, polymethacrylate, polymethylmethacrylates, polyacrylamides and their cross-linked derivatives, cellulose derivatives such as methyl cellulose, hydroxyethyl cellulose, hydroxypropylmethyl cellulose, and hydroxypropyl cellulose, sorbitol (glucitol), naturally derived gums such as xanthan gum, acacia gum, carob gum; alginate derivatives, pectin, carbomer, trolamine, glycerine and polysorbate.

The thickener is incorporated in an amount suitable to obtain the desired thickening effect.

Additionally, other ingredients such as but not limited to antioxidants, pH modifiers, perfumes, preservatives, and colours may be included within the formulation.

In one embodiment, the vasodilator formulation may be delivered using the following non limiting examples: carbomer gel, serum, spray, bath oils, massage oils, patches, nanopatches, nanodelivery systems, compresses, poultices, tape, bandages, strapping tape, dose delivery devices, slow release devices, epidermal injection, subcutaneous injection, dropper, clothing, dressings, gauze and/or injection.

### Method of Production

In an aspect, the invention provides a method of producing the topical formulation disclosed herein. In one embodiment, the method includes the step of micronizing at least one of the dry ingredients. Preferably, the micronized ingredients are combined with a wetting agent or soluble carrier, before mixing with the remaining ingredients to produce the topical vasodilator formulation.

In one embodiment, micronization of the dry ingredients is achieved through milling, bashing, macerating and/or grinding.

In one embodiment, the dry ingredient is at least one arginine or derivatives thereof. The dry ingredient may be one or more ecdysterones.

In one embodiment, micronization of the dry ingredients is achieved through the following non-limiting examples: milling bashing, maceration and/or grinding, mechanical impact mills (e.g., hammer mills and pin mills), fluid energy mills (e.g., spiral jet mills, pancake mills, loop jet mills or fluidized bed jet mills), RESS (Rapid Expansion of Supercritical Solutions) process, the SAS (Supercritical Anti-Solvent) method and the PGSS method (Particles from Gas Saturated Solutions) and Nanoparticalization.

The micronized dry ingredients may be combined with the remaining ingredients in any order to form the vasodilator formulation. Preferably, the micronized dry ingredients are combined with a wetting agent or soluble carrier, prior to being combined with the remaining ingredients. Optionally, the black pepper and/or peppermint extract or one or more components or derivatives thereof may be added last to the formulation.

In a particular embodiment the method includes heating stearic acid to form a liquid to which is added a vitamin E liquid. The mixed stearic acid and vitamin E liquid may be combined with the liquid phase (i.e water-soluble) ingredients. This may then be mixed in an emulsifying mixer. Emulsifying ointment may be added and mixing continued until lump free. This embodiment may further include the step of dissolving an ecdysteroid (e.g. micronized cyanotis extract) in ethanol. The cyanotis extract and ethanol may then be added to the emulsifying mixing drum and blended until smooth and of consistent color. The remaining essential oils and preservative ingredients are combined and then added to the emulsifying mixing drum and continue to blend until smooth and consistent. This particular embodiment preserves the potency of the essential oils, Preserving the essential oil potency is assisted by avoiding the exposure to heat by only heating the stearic acid to make it liquid. Combining other ingredients at room temperature will improve the efficacy of this formulation.

### Methods of Treatment and Use of the Topical Formulation

The topical formulation of the invention finds use in increasing, enhancing and/or stimulating blood flow and/or vasodilation in a subject to assist in the therapeutic treatment of diseases and/or conditions such as for example diabetes and in cosmetic applications such as for example, the enhancement of muscle and vein definition in a body builder, physique model, model and/or an aesthetically conscious individual.

In a third aspect, the invention provides a method of treating or preventing a disease, disorder or condition associated with a decrease and/or impairment of blood flow and/or vasodilation in a subject, said method including the step of topically administering to said subject an effective amount of a topical vasodilator formulation according to the first aspect or produced according to the method of the second aspect, to increase, enhance and/or stimulate blood flow and/or vasodilation in the subject.

In a fourth aspect, the invention provides the use of a topically administrable vasodilator formulation according to the first aspect or produced according to the method of the second aspect for the therapeutic and/or prophylactic treatment of a disease, disorder or condition associated with a decrease and/or impairment of blood flow and/or vasodilation in a subject.

In one embodiment, the topical vasodilator formulation is topically administered to a subject at the site of or at a location proximal to said decrease and/or impairment of blood flow and/or vasodilation.

The terms “administration” or “administered” describe the introduction of the topical vasodilator formulation, to a subject's skin.

The term “therapeutically effective amount” describes a quantity of the formulation of the first aspect or the formulation produced according to the method of the second aspect to achieve a desired effect in a subject being treated with the formulation. For example, this can be the amount of the formulation necessary to increase, enhance and/or stimulate blood flow or vasodilation in a subject. In some embodiments, a “therapeutically effective amount” is sufficient to treat the vasoconstriction or decreased or impaired blood flow entirely. In other embodiments, a “therapeutically effective amount” is an amount sufficient to achieve an enhancement or increased stimulation of blood flow and/or vasodilation in a subject.

Ideally, a therapeutically effective amount of the vasodilator formulation is an amount sufficient to induce the desired result without causing a substantial cytotoxic effect in the subject. The effective amount of the formulation of the first aspect or produced according to the method of the second aspect, useful for increasing, enhancing and/or stimulating blood flow in a subject will be dependent on the subject being treated, the type and severity of any associated disease, disorder and/or condition, and the manner of administration of the vasodilator formulation,

By “increasing”, “enhancing” or “stimulating” as in increasing, enhancing or stimulating vasodilation and blood flow in a subject, is meant a widening of the blood vessels and an increased amount of blood flowing through the blood vessels of a subject. Such an increase need not restore the subject to their full health to be beneficial to the subject. Blood flow and vasodilation in a subject can be determined using any methods or standards known to the ordinarily skilled artisan, including both qualitative and quantitative methods and standards.

A “prophylactic” treatment is a treatment administered to a subject who does not exhibit signs of vasoconstriction or impaired blood flow and exhibits only early signs for the purpose of decreasing the risk of developing vasoconstriction.

In practicing the methods of the invention, the topical formulation may be administered to any topical site on a subject. Topical sites of interest include without limitation: arms, legs, feet, hands, torso, head, etc. The surface area that is covered by the topical formulation following application must be sufficient to provide for the desired amount of formulation to provide vasodilating properties.

In one embodiment, the period of time that the topical vasodilator formulation is maintained at the site of application is about 48 hours. In a further embodiment, the time that the topical vasodilator formulation is maintained at the site of application is about 24 hours.

Suitably, the period of time during; which the formulation is maintained at the application site is at least about 1, 2, 3, 4, 5, 10, 15, 20, 30, 45, 50 minutes, 1, 1.5, 2, 3, 3.5, 4, 5, 6, 7, 8, 9, 10, 12, 15, 20, 24, 48 or 72 hours. Suitably, the formulation is maintained at the site for as long as required by the subject to increase, enhance and/or stimulate vasodilation and blood flow.

In one embodiment, a given dosage of the topical vasodilation formulation may be applied as a single application or a plurality of applications over a given time period, e.g., for as long as the subject requires treatment, where the dosing schedule is administered over a given time period, examples of which include hourly, daily, weekly, biweekly or monthly dosing schedules.

In one embodiment, the formulation application site may be at multiple locations on a subject. The application site to which the topical formulation is applied will be sufficiently proximal to the body area on a subject that requires increased or enhanced blood flow, so that upon application of the formulation, the components of the formulation can readily reach the affected site and actively work to enhance vasodilation of the blood vessels and increase, enhance and/or stimulate blood flow.

Suitably, the vasodilator formulation is generally applied by the subject for a period of time sufficient for the desired amount of vasodilation to be achieved.

If a reduction in blood flow and/or vasodilation occurs following removal or non-use of the topical vasodilator formulation, further topical formulation may be applied. The process may be repeated as necessary and when desired by the subject to achieve effective vasodilation.

In some embodiments, the patient may experience vasodilation and/or enhanced blood flow either immediately or shortly after application. Suitably, the patient will experience at least sonic vasodilation effects about 1 to 60 seconds; 1, 2, 3, 4, 5, 6, 7, 8, 9, 10, 15, 20, 30, 40, 50, 60 minutes; or 1, 1.5, 2, 2.5, 3, 4, 5, 6, 7, 8, 9, 10, 15, 20, 25 hours or days following application of the topical formulation.

The amount of topical vasodilator formulation applied will usually be sufficient to cover the area of skin overlying the site of reduced blood flow so that the subject experiences vasodilation and an increase or enhanced blood flow. For solutions, liquids, gels, lotions, creams, ointments and the like, the topical formulation may be applied to the subject at a desired skin site and a covering optionally applied thereto. For example, such covering may include patches, bandages, plasters and dressings. An appropriate sized covering may be placed over the applied topical vasodilator formulation. Conveniently, the topical formulation may be provided in a unit dosage dispenser, such as for example a pump bottle, spray, dropper or roll-on device examples of which are well known in the art.

Upon application of the topical formulation, the components of the formulation penetrate the surface of the skin and the subject experiences vasodilation and increased blood flow. Suitably, the subject experiences at least a partial increase or enhancement of blood flow. Preferably, the subject experiences restored blood flow and in some cases may experience a complete restoration of blood flow. Accordingly, application of the topical formulation in accordance with the methods of the invention results in treatment of the subject suffering from vasoconstriction and/or impairment of blood flow.

The topical formulation of the invention is available to a plurality of subjects. The term “subject” is used in its broadest sense. In a preferred embodiment, the subject is a mammal. More preferably, the subject is a human. Non-limiting examples of mammals include humans, dogs, cats, horses, cows, sheep, goats and pigs. Preferably, a subject includes any human or non-human mammal, including for example, a primate, cow, horse, pig, sheep, goat, dog, cat, Or rodent.

By “treatment” is meant at least an amelioration of the vasoconstriction or reduced blood flow experienced by a subject, where amelioration is used in a broad sense to refer to at least a reduction in the magnitude of a parameter, e.g. vasoconstriction, associated with the condition, disorder or disease being treated. As such, treatment also includes situations where the vasodilation and/or blood flow is completely restored, e.g., such that the host no longer suffers from vasoconstriction or reduced blood flow. The beneficial effect of the vasodilator formulation can be determined using any methods or standards known to a person of skill in the art.

The topical vasodilator formulation of the invention may be used to treat vasoconstriction and/or reduced blood flow associated with many conditions by topically applying the formulation to the effected site as described in the aforementioned embodiments. Specifically, the topical formulation may be used to treat vasoconstriction and/or reduced blood flow, including, but not limited to: erectile dysfunction, aging, baldness, peripheral neuropathy, microangiopathy, female sexual dysfunction, male sexual dysfunction, diabetes, aging, restless leg syndrome, raynaud's phenomenon, Buerger's Disease, chilblains, numbness and tingling of extremities, varicose veins, haemorrhoids, hypothyroidism, immobility, cellulite, accumulation of subcutaneous adipose tissue, cosmetic applications such as poor quality hair, nail and skin, lymphedema, swelling of the hands and feet, oedema, deep vein thrombosis, ischemia, chronic venous insufficiency, gangrene, vasoconstriction, thrombosis, emboli sin, paraesthesia, poikilothermia, cellulites, tissue necrosis, ischaemic neuropathy, leg cramps either idiopathic, or related to either pregnancy, renal dialysis, or peripheral vascular disease (both venous and arterial), or to revitalize muscle, or to improve muscle strength and recovery after vigorous exercise and intense sport, or to improve muscle strength and performance during vigorous exercise and intense sport.

### Method of Enhancing Muscular and/or Vascular Definition

In a fifth aspect, the invention provides a method of enhancing muscular and/or vascular definition in a subject, the method including the step of topically administering to said subject an effective amount of a topical vasodilator formulation according to the first aspect or produced according to the method of the second aspect, to enhance muscular and/or vascular definition in the subject.

In one embodiment the subject is a body builder, physique model, model and/or aesthetically conscious individual.

In one embodiment, the vasodilator formulation may be applied to one or more target sites on a subject for a period of time to achieve a desired enhancement of muscular and/or vascular definition. Suitably, the vasodilator formulation may be applied to one or more target sites on a subject for up to 60 minutes prior to exercise, during exercise, or up to 60 minutes post exercise.

In one embodiment, for example, the vasodilator formulation may be applied to one or more target sites on a subject prior to modelling, physique and/or body building competitions or presentations.

In one embodiment, the vasodilator formulation may be applied to one or more target sites on a subject that wishes to improve and/or enhance muscular and/or vascular definition.

### Kits

In a sixth aspect, the invention provides a kit comprising the topically administrable vasodilator formulation according to the first aspect or produced according to the method of the second aspect, an applicator device and instructions for using said formulation to increase, enhance and/or stimulate blood flow and/or vasodilation in a subject.

The applicator device can be any device that ensures correct and targeted delivery of the vasodilator formulation of the first aspect or produced according to the method of the second aspect. Examples of appropriate devices include applicators that attach to a single- or multi-dose container of the formulation, tubes, roll-on device, brush, sponges, spray, aerosol, syringe or droppers.

Preferably, the applicator device is a pump device.

The kit may also include instructions for how to use the formulation, where the instructions typically include information about how to apply the formulation, dosing schedules etc. The instructions are generally recorded on a suitable recording medium. For example, the instructions may be printed on a substrate, such as paper or plastic, etc. As such, the instructions may be present in the kits as a package insert, in the labelling of the container of the kit or components thereof (i.e associated with the packaging or sub packaging) etc. In other embodiments, the instructions are present as an electronic storage data file.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

### EXAMPLES

The topical vasodilator formulations may be provided as an oil, mousse, gels, creams, lotions, balms, foams, liquids, aerosols, self-tanning lotion, spray or paint and the like.

The following examples and accompanying tables provide embodiments of the vasodilator formulations of the invention, methods for preparing same and methods of administration to a subject.

### Example 1

A topical vasodilator formulation was prepared comprising 2.5% Arginine Hcl, 0.25% Black Pepper Essential oil and 0.25% Peppermint essential oil as per the following formulation (Table 1) to provide a formulation suitable to be used for example in a pump device.

The formulation may be used to enhance muscular and/or vascular definition in a subject, in particular a subject practicing body building.

Approximately, 5% Cyanotis vaga extract supplying 50% 20-Hydroxyecdysterone and 2.5% arginine Hcl was micronized to form a powder and combined with the wetting agent ethanol. In a separate container, the base was formed through heating the oil phase in a container to 60 degrees and then heating the water phase in a separate container to 60 degrees. Both the oil and water phase were then combined to form an emulsion. The micronized powders 5% Cyanotis vaga extract supplying 50% 20-Hydroxyecdysterone and 2.5% arginine Hcl are added to the base using a geometric dilution. The black pepper and peppermint essential oils were further combined, with the formulation in an ointment mill to form an active blend.

Optionally, the formulation may comprise alterative ecdysterones.

The formulation may then be transferred to a suitable application device such as for example a pump device, which allows for an easy, no mess application by a subject.

### Example 2

A topical vasodilator formulation was prepared comprising 2.5% Arginine Hcl, 0.25% Black Pepper Essential oil, 0.25% Peppermint essential oil as per the following formulation (Table 2) to provide a formulation suitable to be used for example in a pump device.

The formulation may be used in the treatment of a disease, disorder or condition associated with a decrease and/or impairment of blood flow in a subject, in particular targeting peripheral blood flow, suitably diabetes.

Approximately, 5% Cyanotis vaga extract supplying 50% 20-Hydroxyecdysterone and 2.5% arginine Hcl was micronized to form a powder and combined with the wetting agent ethanol. The base was formed through heating the oil phase in a container to 60 degrees and then heating the water phase in a separate container to 60 degrees. Both the oil and water phase were then combined to form an emulsion. The micronized powders of 5% Cyanotis vaga extract supplying 50% 20-Hydroxyecdysterone and 2.5% arginine Hcl to the base using a geometric dilution were combined. Black pepper and peppermint essential oil were combined with the mixture in an ointment mill to form an active blend.

Optionally, the formulation may further comprise an antimicrobial antiseptic, such as for example Kunzea ambigua extract or a component or derivative thereof and Tea tree oil extract or a component or derivative thereof, or an alternative ecdysteroid may be added to the vasodilator formulation.

The formulation may then be transferred to a suitable application device such as for example a pump device, which allows for an easy, no mess application.

### Example 3

A topical vasodilator formulation was prepared comprising 2.5% Arginine Hcl, 0.25% Black Pepper Essential oil and 0.25% Peppermint essential oil as per the following formulation (Table 3) to provide a formulation suitable to be used for example in a pump device.

The formulation may be used in the treatment of a disease, disorder or condition associated with a decrease and/or impairment of blood flow in a subject, in particular to treat erectile dysfunction and female sexual dysfunction.

Approximately, 5% Cyanotis vaga extract supplying 50% 20-Hydroxyecdysterone and 2.5% arginine Hcl was micronized to form a powder and combined with the wetting agent ethanol. The base was formed through heating the oil phase in a container to 60 degrees and then heating the water phase in a separate container to 60 degrees. Both the oil and water phase were then combined to form an emulsion. The micronized powders of 5% Cyanotis vaga extract supplying 50% 20-Hydroxyecdysterone and 2 5% arginine Hcl to the base using a geometric dilution were combined. Black pepper and peppermint essential oil were combined with the mixture in an ointment mill to form an active blend.

Optionally, the formulation may further comprise a PDE inhibitor, Quercetin or derivatives thereof, alpha-2 adrenoceptor antagonist yohimbine Hcl or derivatives thereof, Lysine or a lubricant or an ingredient such as glow in the dark compounds that may be added to the vasodilator formulation.

The formulation may then be transferred to a suitable application device such as for example a pump device, which allows for an easy, no mess application.

### Example 4

A topical vasodilator formulation was prepared comprising 2.5% Arginine Hcl, 0.25% Black Pepper Essential oil and 0.25% Peppermint essential oil as per the following formulation (Table 4) to provide a formulation suitable to be used for example in a pump device.

The formulation may be used in the treatment of a disease, disorder or condition associated with a decrease and/or impairment of blood flow in a subject, in particular to treat hair loss and/or hair regrowth.

Approximately, 5% Cyanotis vaga extract supplying 50% 20-Hydroxyecdysterone and 2.5% arginine Hcl was micronized to form a powder and combined with the wetting agent ethanol. The base was formed through heating the oil phase in a container to 60 degrees and then heating the water phase in a separate container to 60 degrees. Both the oil and water phase were then combined to form an emulsion, The micronized powders of 5% Cyanotis vaga extract supplying 50% 20-Hydroxyecdysterone and 25% arginine Hcl to the base using a geometric dilution were combined. Black pepper and peppermint essential oil were combined with the mixture in an ointment mill to form an active blend.

Optionally, the formulation may further comprise Saw Palmetto extract—5-alpha reductase inhibitors 2.5%, Eclipta alba 2.5%, Crinum asiaticum 2.5%, Polypodium leucotomas 2.5%, L-ascorbate 2-phosphate—5% (or 1-ascorbate, ascorbic acid, ascorbic acid-2-phosphate, or any other form of ascorbates salt), niacinamide 5% nicotinic acid, methyl nicotinate, Sea buckthorn oil or omega 7 oil may be added to the vasodilator formulation.

The formulation may then be transferred to a suitable application device such as for example a pump device, which allows for an easy, no mess application.

### Example 5

A topical vasodilator formulation was prepared comprising 2.5% Arginine Hcl, 0 25% Black Pepper Essential oil and 0.25% Peppermint essential oil as per the following formulation (Table 5) to provide a formulation suitable to be used for example in a pump device.

The formulation may be used in the treatment of a disease, disorder or condition associated with a decrease and/or impairment of blood flow in a subject, in particular to treat aging.

Approximately, 5% Cyanotis vaga extract supplying 50% 20-Hydroxyecdysterone and 2.5% arginine Hcl was micronized to form a powder and combined with the wetting agent ethanol. The base was formed through heating the oil phase in a container to 60 degrees and then heating the water phase in a separate container to 60 degrees. Both the oil and water phase were then combined to form an emulsion. The micronized powders of 5% Cyanotis vaga extract supplying 50% 20-Hydroxyecdysterone and 2.5% arginine Hcl to the base using a geometric dilution were combined. Black pepper and peppermint essential oil were combined with the mixture in an ointment mill to form an active blend.

Optionally, the formulation may further comprise Bioidentical hormones including for example: testosterone, progesterone, estradiol, estrone, estriol, pregnenelone, human chorionic gonadotropin (hCG), Dehydroepiandrosterone (DHEA) and peptides GHRP-6, CJC1295, AOD9604 or derivatives thereof.

The formulation may then be transferred to a suitable application device such as for example a pump device, which allows for an easy, no mess application.

### Example 6

An alternative embodiment of a method of formulation is provided in this Example. It is envisaged that method according to the embodiment may provide a particularly stable and more effective formulation at a cheaper cost. By way of Example, the method may be performed generally with reference to the components set forth in Table 6.

The method includes the following steps
1. Add water phase ingredients to emulsifying mixer drum and mix until dissolved
2. Dissolve Cyanotis extract in ethanol in separate container
3. In a separate container heat stearic acid to make liquid and add vitamin E to liquid stearic acid
4. add step 3 to step 1 in emulsifying mixer
5. add emulsifiers to mixer and blend until smooth with no lumps
6. Add step 2 to step 5 and blend until consistent color and smooth texture
7 combine preservatives and essential oils in a separate container
7. add step 7 and step 6 in emulsifying mixer
8. Continue to mix until blended

The cyanotis extract and arginine HCl are micronized prior to their inclusion as previously described. Combine water phase ingredients demineralised water, Arginine Hcl, Aloe Barbadensis Leaf Juice, EDTA, Sorbitol solution, glycerine liquid BP/USP and Propylene Glycol into drum and blend until dissolved. In a separate container dissolve cyanotis extract in ethanol. In a separate container heat stearic acid to make liquid and then add the vitamin E liquid. Combine the liquid stearic acid and vitamin E mix into the water phase ingredients whilst mixing in the emulsifying mixer. Add the emulsifying ointment and continue to mix until lump free. Add the dissolved cyanotis extract and ethanol to the emulsifying mixing drum and continue to blend until smooth and consistent color. Combine the remaining essential oils and preservative ingredients in a separate container. Add the essential oils and preservatives to the emulsifying mixing drum and continue to blend until smooth and consistent.

The formulation may then be transferred to a suitable application device such as for example a pump device, which allows for an easy, no mess application.

Table 7 provides a specific embodiment of a rosemary oil-containing formulation that may be produced according to the method described in this Example. The method preserves the potency of the essential oils, such as on rosemary-containing formulation but also for other formulations disclosed herein Preserving the essential oil potency is assisted by avoiding the exposure to heat by only heating the stearic acid to make it liquid and combining all other ingredients at room temperature will improve the efficacy of the formulation.

Throughout the specification the aim has been to describe the preferred embodiments of the invention without limiting the invention to any one embodiment or specific collection of features. It will therefore be appreciated by those of skill in the art that in light of the instant disclosure, various modifications and changes can be made in the particular embodiments exemplified without departing from the scope of the present invention.

TABLE 1

| Application | Body Building |
|---|---|
| Ingredients | Arginine Hcl 2.5% (0.1% to 75%) |
| | Ethanol (to wet dry ingredients) |
| | Aqua |
| | Natural Emulsifying wax (Cetearyl alcohol and |
| | Ceteareth-20) |
| | Ethyl-alcohol, Sorbitol |
| | Aloe Barbadensis Leaf Juice |
| | Stearic acid |
| | Cyclopentasiloxane |
| | Tocopheryl acetate (Vitamin E) |
| | Black Pepper Essential oil |
| | Peppermint essential oil |
| | Phenoxyethanol |
| | Caprylyl Glycol |
| Optional Ingredients | Cyanotis vaga extract or components or |
| | derivatives thereof |
| | Phytoecdysterones standardised to 50% |
| | 20-Hydroxyecdysterone at 5% (1% to 10%) or |
| | alternative ecdysteroids |
| Functional Properties of | Vasodilation |
| each formulation | Increased protein synthesis |
| | Anti-catabolic |

TABLE 2

| Application | Peripheral Circulation (diabetes) |
|---|---|
| Ingredients | Arginine Hcl 2.5% (0.1% to 75%) |
| | Ethanol (to wet dry ingredients) |
| | Aqua |
| | Natural Emulsifying wax (Cetearyl alcohol and |
| | Ceteareth-20) |
| | Ethyl-alcohol, Sorbitol |
| | Aloe Barbadensis Leaf Juice |
| | Stearic acid |
| | Cyclopentasiloxane |
| | Tocopheryl acetate (Vitamin E) |
| | Black Pepper Essential oil |
| | Peppermint essential oil |
| | Phenoxyethanol |
| | Caprylyl Glycol |
| Optional Ingredients | Antimicrobial antiseptic(s): Kunzea ambigua |
| | oil, Tea tree oil |
| | Cyanotis vaga extract standardised to 50% |
| | 20-Hydroxyecdysterone at 5% (1% to 10%) or |
| | alternative ecdysteroids |
| Functional Properties of | Vasodilation |
| each formulation | Increased wound healing |
| | Lower blood glucose |

TABLE 3

| | |
|---|---|
| Application | Erectile Dysfunction and female sexual |
| | dysfunction |
| Ingredients | Arginine Hcl 2.5% (0.1% to 75%) |
| | Ethanol (to wet dry ingredients) |
| | Aqua |
| | Natural Emulsifying wax (Cetearyl alcohol and |
| | Ceteareth-20) |
| | Ethyl-alcohol, Sorbitol |
| | Aloe Barbadensis Leaf Juice |
| | Stearic acid |
| | Cyclopentasiloxane |
| | Tocopheryl acetate (Vitamin E) |
| | Black Pepper Essential oil |
| | Peppermint essential oil |
| | Phenoxyethanol |
| | Caprylyl Glycol |
| Optional Ingredients | PDE inhibitors - Quercetin |
| | Alpha-2 adrenoceptor antagonists - Yohimbine |
| | Hcl |
| | Lysine - reduce risk of arginine flare of herpes |
| | virus in predisposed/infected individuals |
| | Alternative base can be used to work as a |
| | “lubricant” |
| Functional Properties of | Vasodilation |
| each formulation | PDE4 and PDE5 inhibition |
| | Reduce risk of HPV flare and subsequent |
| | spread |
| | Topical cream and/or lubricant to enhance |
| | sexual performance in males and females |

TABLE 4

| | |
|---|---|
| Application | Hair Loss/hair regrowth |
| Ingredients | Arginine Hcl 2.5% (0.1% to 75%) |
| | Ethanol (to wet dry ingredients) |
| | Aqua |
| | Natural Emulsifying wax (Cetearyl alcohol and |
| | Ceteareth-20) |
| | Ethyl-alcohol, Sorbitol |
| | Aloe Barbadensis Leaf Juice |
| | Stearic acid |
| | Cyclopentasiloxane |
| | Tocopheryl acetate (Vitamin E) |
| | Black Pepper Essential oil |
| | Peppermint essential oil |
| | Phenoxyethanol |
| | Caprylyl Glycol |
| Optional Ingredients | Saw Palmetto extract - 5 alpha reductase |
| | inhibitors 2.5% (0.5% to 25%) |
| | Eclipta alba 2.5% - (0.5% to 25%) |
| | Crinum asiaticum 2.5% - (0.5% to 25%) |
| | Polypodium leucotomas 2.5% - (0.5% to 25%) |
| | L-ascorbate 2-phosphate - 5% (or 1-ascorbate, |
| | ascorbic acid, ascorbic acid-2-phosphate, or |
| | any other form of ascorbates salt) |
| | (0.1% to 20%) |
| | niacinamide 5% nicotinic acid, methyl |
| | nicotinate (0.1% to 10%) |
| | Sea buckthorn oil - omega 7 oil (0.5% to 10%) |
| Functional Properties of | Vasodilation |
| each formulation | Reduce hair loss |
| | Enhance Hair regrowth |
| | Improve health of scalp |
| | Suitable for male and female pattern baldness |
| | and all forms of alopecia |

TABLE 5

| | |
|---|---|
| Application | Anti-aging |
| Ingredients | Arginine Hcl 2.5% (0.1% to 75%) |
| | Ethanol (to wet dry ingredients) |
| | Aqua |
| | Natural Emulsifying wax (Cetearyl alcohol and |
| | Ceteareth-20) |
| | Ethyl-alcohol, Sorbitol |
| | Aloe Barbadensis Leaf Juice |
| | Stearic acid |
| | Cyclopentasiloxane |
| | Tocopheryl acetate (Vitamin E) |
| | Black Pepper Essential oil |
| | Peppermint essential oil |
| | Phenoxyethanol |
| | Caprylyl Glycol |
| Optional Ingredients | Bioidentical hormones |
| Functional Properties of | Vasodilator and penetration enhancers to |
| each formulation | increase the absorption and utilization of |
| | bio-identical hormones |

TABLE 6

water phase
Demineralised water
Arginine Hcl (2.5%)
Aloe Barbadensis Leaf Juice
EDTA
Sorbitol solution
glycerine liquid BP/USP
Propylene Glycol
oil phase
Stearic acid
Tocopheryl acetate (Vitamin E) liquid BP/USP
emulsifiers
Emulsifying Wax
White Soft Paraffin
Liquid Paraffin
preservatives and essential oils
dimethicone 100cs
Cyclomethicone
Rosemary essential oil
Black Pepper Essential oil (0.25%)
Pepperment essential oil (0.1%)
phenoxyethanol
caprylyl glycol

TABLE 7

| | | |
|---|---|---|
| Cyanotis vaga extract | 3 | g |
| (50% 20-Hydroxyecdysterone) | | |
| Arginine Hcl | 1.5 | g |
| Black Pepper Essential oil | 0.15 | ml |
| Peppermint essential oil | 0.06 | ml |
| Rosemary essential oil | 5 | ml |
| Demineralised water | 32.6 | ml |
| Emulsifying Wax | 5 | g |
| White Soft Paraffin | 8.45 | g |
| Liquid Paraffin | 3.3 | ml |
| Ethyl-alcohol | 6.6 | ml |
| Sorbitol solution | 1.36 | ml |
| Aloe Barbadensis Leaf Juice | 1.36 | ml |
| Stearic acid | 1.36 | g |
| Cyclomethicone | 0.27 | ml |
| dimethicone 100cs | 0.27 | ml |
| Tocopheryl acetate (Vitamin E) liquid BP/USP | 0.27 | ml |
| glycerine liquid BP/USP | 10.86 | ml |
| Propylene Glycol | 1.35 | ml |
| phenoxyethanol | 0.396 | ml |
| caprylyl glycol | 0.264 | ml |
| EDTA | 0.014 | g |

1. A topically administrable vasodilator formulation comprising:
(i) arginine and/or one or more derivatives thereof;
(ii) black pepper extract and/or one or more components or derivatives thereof; and
(iii) peppermint extract and/or one or more components or derivatives thereof.

2. The topical pharmaceutical formulation of claim 1, wherein arginine is selected from the group consisting of: 2-Amino-5-guanidinopentanoic acid, agmatine, arginine hydrochloride, Ark 1, decarboxylated arginine, dipeptide arginyl aspartate, D-arginine, L-arg, L-arginine, L-arginine aspartate, NG-monomethyl-L-arginine, arginine alpha ketogluterate, arginine ethyl esther, norvaline, arginine salt, arginine ester, Argivene, Detoxargin, Levargin, Minophagen Argamine, Polyarginine, Arginina, (S)-2-Amino-5-guanidinopentanoic acid, 2-amino-5-guanidinovaleric acid, Argininum [INN-Latin], Arginina [INN-Spanish], L-alpha-Amino-delta-guanidinovaleric acid, L-Arginin, Poly(L-arginine), L-Ornithine, N5-(aminoiminomethyl)-, L(+)-Arginine, 1-Amino-4-guanidovaleric acid, H-Arg-OH, L-a-Amino-d-guanidinovaleric acid, L-Arginine, homopolymer, (S)-2-Amino-5-[(aminoiminomethyl)amino]pentanoic acid, L-Arginine hydrochloride, L-Norvaline, 5-((aminoiminomethyl)amino)-, (S)-2-Amino-5-guanidinovaleric acid, (2S)-2-amino-5-(diaminomethylideneamino)pentanoic acid, (S)-2-Amino-5-((aminoiminomethyl)amino)pentanoic acid, Pentanoic acid, 2-amino-5-((aminoiminomethyl)amino, L-Norvaline, 5-[(aminoiminomethyl)amino]- 2-Amino-5-Guanidnovaleric Acid, Argininum Pentanoic acid, 2-amino-5-[(aminoiminomethyl)amino]-, Tocris-0663, L-Arginine (JP16), Lopac-A-5006, Arginine, 2-amino-5-guanidinovalerate, Arginine hydrochloride (USAN), L-a-Amino-d-guanidinovalerate, N5-(aminoiminomethyl)-L-Ornithine, L-alpha-Amino-delta-guanidinovalerate, (2S)-2-amino-5-guanidinopentanoic acid, 5-[(aminoiminomethyl)amino]-L-Norvaline, (2S)-2-amino-5-(carbamimidamido)pentanoic acid, L-Arginine-L-Glutamate 2-Amino-Pentanedoic Acid, (S)-2-amino-5-[(aminoiminomethyl)amino]-Pentanoate, (S)-2-amino-5-[(aminoiminomethyl)amino]-Pentanoic acid, (2S)-2-azanyl-5-[bis(azanyl)methylideneamino]pentanoic acid, alpha-keto-gamma-guanidovaleric acid, ornithine, citrulline, guanidoacetic acid and ornithine.

3. The vasodilator formulation of claim 1, wherein the formulation comprises from about 0.1% to about 75% of arginine and/or one or more derivatives thereof.

4. The vasodilator formulation of claim 1, wherein the formulation comprises about 2.5% arginine and/or one or more derivatives thereof.

5. The vasodilator formulation of claim 1, wherein the formulation comprises from about 0.01% to about 25% black pepper extract and/or one or more components or derivatives thereof.

6. The vasodilator formulation of claim 1, wherein the formulation comprises about 0.25% black pepper extract and/or one or more components or derivatives thereof.

7. The vasodilator formulation of claim 1, wherein the formulation comprises from about 0.01% to about 25% peppermint extract and/or one or more components or derivatives thereof.

8. The vasodilator formulation of claim 1, wherein the formulation comprises from about 0.25% peppermint extract and/or one or more components or derivatives thereof.

9. The vasodilator formulation of claim 1, wherein the formulation further comprises rosemary oil and/or one or more components or derivatives thereof.

10. The vasodilator formulation of claim 1, wherein the formulation further comprises one or more penetration enhancers.

11. The vasodilator formulation of claim 10, wherein the penetration enhancer is Aloe vera extract and/or stearic acid.

12. The vasodilator formulation of claim 1, further comprising at least one pharmaceutically acceptable carrier, diluent and/or excipient.

13. The vasodilator formulation of claim 12, wherein the pharmaceutically acceptable carrier is an oil-in-water emulsion.

14. The vasodilator formulation of claim 1, further comprising at least one terpene.

15. The vasodilator formulation of claim 14, wherein the terpene is black pepper extract or one or more components or derivatives thereof.

16. The vasodilator formulation of claim 1, further comprising an ecdysteroid.

17. The vasodilator formulation of claim 1, further comprising an antimicrobial.

18. The vasodilator formulation of claim 1, further comprising an alpha-2 antagonist.

19. The vasodilator formulation of claim 1, further comprising a phosphodiesterase-5a inhibitor.

20. The vasodilator formulation of claim 1, further comprising lysine.

21. The vasodilator formulation of claim 1, further comprising a 5-alpha reductase inhibitor.

22. The vasodilator formulation of claim 1, further comprising a hair growth promoter.

23. The vasodilator formulation of claim 1, further comprising at least one immune and/or cytokine modulator.

24. The vasodilator formulation of claim 1, further comprising a local vasodilator.

25. The vasodilator formulation of claim 1, further comprising a Hippophae rhamnoide extract or one or more components or derivatives thereof.

26. The vasodilator formulation of claim 1, wherein the formulation is an oil, mousse, gel, cream, lotion, balm, lubricant, foam, liquid or aerosol, self-tanning lotion, oil, spray or paint.

27. A method of producing the topically administrable vasodilator formulation according to claim 1, including the step of combining arginine and/or one or more derivatives thereof; black pepper extract and/or one or more components or derivatives thereof; and peppermint extract and/or one or more components or derivatives thereof to thereby produce the topically administrable vasodilator formulation.

28. A method of treating or preventing a disease, disorder or condition associated with a decrease and/or impairment of blood flow in a subject, said method including the step of topically administering to said subject an effective amount of a topical vasodilator formulation according to claim 1, to increase, enhance and/or stimulate blood flow and or vasodilation in the subject.

29. A topically administrable vasodilator formulation according to claim 1, for therapeutic and/or prophylactic treatment of a disease, disorder or condition associated with a decrease and/or impairment of blood flow and/or vasodilation in a subject.

30. The method of claim 28, wherein the disease, disorder or condition associated with a decrease and/or impairment of blood flow is selected from the group consisting of: erectile dysfunction, aging, baldness, peripheral neuropathy, microangiopathy, female sexual dysfunction, male sexual dysfunction, diabetes, aging, restless leg syndrome, raynaud's phenomenon, Buerger's Disease, chilblains, numbness and tingling of extremities, varicose veins, haemorrhoids, hypothyroidism, immobility, cellulite, accumulation of subcutaneous adipose tissue, cosmetic applications such as poor quality hair, nail and skin, lymphedema, swelling of the hands and feet, oedema, deep vein thrombosis, ischemia, chronic venous insufficiency, gangrene, vasoconstriction, thrombosis, embolism, paraesthesia, poikilothermia, cellulites, tissue necrosis, ischaemic neuropathy, leg cramps either idiopathic, or related to either pregnancy, renal dialysis, or peripheral vascular disease (both venous and arterial), or to revitalize muscle, or to improve muscle strength and recovery after vigorous exercise and intense sport, or to improve muscle strength and performance during vigorous exercise and intense sport.

31. The method of claim 28, wherein the subject is a human.

32. A method of enhancing muscular and/or vascular definition in a subject, the method including the step of topically administering to said subject an effective amount of a topical vasodilator formulation of claim 1, to enhance muscular and/or vascular definition in the subject.

33. The method of claim 32, wherein the subject is a body builder, physique model, model and/or an aesthetically conscious individual.

34. A kit comprising the vasodilator formulation according to claim 1, an applicator device and instructions for using said formulation to increase, enhance and/or stimulate blood flow and/or vasodilation in a subject.
